(19) United States
(12) Patent Application Publication
(10) Pub. No.: US 2018/0171383 A1
(43) Pub. Date: Jun. 21, 2018

(54) METHODS FOR NUCLEIC ACID IDENTIFICATION

(71) Applicant: Vishal A. Patil, Florence, MA (US)

(72) Inventor: Vishal A. Patil, Florence, MA (US)

(73) Assignee: Niagara Biosciences, Inc., Worcester, MA (US)

(21) Appl. No.: 15/578,870

(22) PCT Filed: Jun. 2, 2016
(86) PCT No.: PCT/US16/35601

§ 371 Date: Dec. 1, 2017

Related U.S. Application Data
(60) Provisional application No. 62/169,585, filed on Jun. 2, 2015.

(51) Int. Cl. C12Q 1/683

(52) U.S. Cl. CPC C12Q 1/683

(57) ABSTRACT

Provided herein are methods and devices for obtaining nucleotide sequence information from nucleic acid and nucleic acid samples. The methods involve modifying and manipulating nucleic acids while in movement (flow) and without reliance on fixation, amplification or hybridization techniques. The methods and devices are sensitive enough to detect signal from and thus interrogate single nucleic acids on an individual basis rather than as a bulk population.

### RELATED APPLICATIONS

This application claims the benefit under 35 U.S.C. § 119(e) of U.S. Provisional Application Ser. No. 62/169,585, filed Jun. 2, 2015, which is incorporated by reference herein in its entirety.

### BACKGROUND OF INVENTION

Nucleic acid analysis, including nucleic acid sequencing, has enabled a diverse range of applications including detection of microorganisms for medical purposes. Existing nucleic acid technologies for identifying microorganisms include pulse gel electrophoresis (PFGE), nucleic acid amplification techniques such as polymerase chain reaction (PCR) and including quantitative reverse transcriptase PCR (RT-qPCR), optical mapping, and hybridization based mapping.

In PFGE, a restriction enzyme (also known as a restriction endonuclease, and referred to herein as RE) that cuts genomic DNA rarely (due to the infrequency of its binding or cleavage site) is used to digest genomic DNA. The digested DNA is then separated in a gel matrix using pulsed electric field. The technique is widely used in epidemiological studies of pathogens. As an example, it is used by the Center for Disease Prevention and Control to identify disease outbreaks. Although it is an established methodology that uses universal reagents, it is labor and time intensive, requiring a laborious sample preparation and yielding results within about 2 days. Additionally, strain typing with the accuracy required is not always possible using a single enzyme digestion. PFGE also cannot handle complex mixtures of pathogens (and thus genomic DNA). Finally, the technique typically requires on the order to 1 microgram of DNA, which may be far more DNA than can be obtained from certain samples,

In PCR, a fragment of DNA or a region within a larger fragment of DNA is amplified and the presence or absence of the product or the sequence of the product is used to determine the presence and identity of the pathogen. This method can be used for complex mixtures of pathogens and DNA because the amplification may be selective (i.e., it may only amplify DNA from a particular pathogen and not genomic DNA from others). It is also highly sensitive, and is able to amplify and thus detect very few copies of starting DNA. It is also easily automated and requires easy sample preparation. It does however require preliminary knowledge of the sequence of the target DNA in order to design primers, and it is dependent on the ability to hybridize the primers to the target DNA. Hybridization may vary between primer pairs, causing certain targets to be amplified to a greater extent than others even though they have been present in the original sample in equal amounts.

Optical mapping is a technique that involves fixing elongated and intercalated DNA fragments to a glass slide and digesting the fixed DNA using REs. Maps of the DNA fragments are then obtained by measuring the gaps (or distances) between the digested fragments. This process, like PFGE, is time intensive with the entire process taking about a week. In addition, the technique does not lend itself to high throughput analysis.

Still other methods involve hybridization of probes to DNA fragments. One such method involves linearizing and fixing DNA in nanochannels, followed by hybridizing the fixed DNA to sequence specific probes. The DNA is then scanned to determine the presence and location of the hybridized probes. The requirement for hybridization of the probes can reduce specificity and introduce artefacts into the ultimate readout.

### SUMMARY OF INVENTION

Provided herein is an alternative approach for nucleic acid analysis that overcomes various limitations of the foregoing methodologies. The approach described herein is able to generate a map or signature of a nucleic acid fragment, without the need for hybridization to probes, or amplification of the original sample, and without any need for fixation (or immobilization) of the nucleic acid. Significantly, it can be performed in a high throughput manner on the order of minutes, thereby facilitating the analysis of samples at a much higher rate than previously possible.

It can be performed on virtually any nucleic acid (e.g., DNA) sample, and requires minimal manipulation of the sample. Accordingly, the samples can be prepared quickly and easily and the analysis itself can be performed within minutes. For example, sample preparation time may be on the order of about 1 hour and analysis time may be on the order of about 15 minutes. The approach also lends itself to automation. Equally significant, this new approach is a single molecule analysis technique that can be performed on as little as a few picograms of DNA as the starting material. This could reduce the time it takes to work up a sample, including reducing the amount of culture needed before isolating the DNA. Further advantages will be described or will become apparent herein.

In its broadest sense, the method involves obtaining sequence information, and thus maps, of nucleic acids such as DNA, by digesting the nucleic acids in a manner such that the digested fragments are analyzed in the order in which they were present in their “parent” nucleic acid and in some instances cleaved from their “parent” nucleic acid. The analysis involves determining the length of the digested fragments, based for example on intercalator brightness. In this way, the readout from the assay is a series of digested fragments each of a particular length (determined by their signal intensity), in an order that is faithful to the order of the fragments in the parent nucleic acid from which they were cleaved. The ability to carry out the analysis, including the entire analysis, in flow (i.e., while the nucleic acid is moving rather than fixed to a solid support) allows for high-throughput analysis.

More specifically, the method involves contacting a parent nucleic acid (such as DNA) with REs of a single type under a condition that allows the RE to bind to but not cleave the nucleic acid (referred to herein as a “binding condition”). An example of such a condition is a solution that contains calcium ions but not magnesium and/or manganese ions (or magnesium and/or manganese ions at a concentration that is insufficient for the RE to cleave the nucleic acid to which it binds or is bound). After incubation with the RE under this condition, the parent nucleic acid is stretched in flow. Stretching of the nucleic acid can be accomplished in a number of ways including by altering sheath fluid flow and/or altering the geometry of the channel through which the parent nucleic acid travels. The nucleic acid can also be stretched by electrophoretic means based on its negative charge.

Once the parent nucleic acid is fully or nearly fully stretched, it is exposed to a condition that allows the RE to cleave the nucleic acid (referred to herein as a “cleaving condition”). An example of a cleaving condition is a solution that contains magnesium and/or manganese ions, which will initiate digestion of the nucleic acid by the bound RE. The cleaving condition can also be an increased concentration of magnesium and/or manganese ions sufficient for the RE to cleave the nucleic acid to which it is bound. These ions can be introduced into the channel through which the parent nucleic acid is travelling for example through the use of porous channel walls or side channels through which sheath fluid and ions travel. In some instances, digestion may occur on single elongated nucleic acids in an ordered manner starting at one end and continuing to the opposite end. In other instances, digestion may occur simultaneously or nearly simultaneously with all the bound RE cutting the parent nucleic acid at the same time or nearly the same time. In still other instances, digestion may occur randomly with bound RE cutting the parent nucleic acid at different times and optionally independently of each other. The parent nucleic acid is maintained in a sufficiently stretched state until digestion is complete (e.g., all the bound RE cut the parent nucleic acid). Maintaining the parent nucleic acid in this sufficiently stretched state enables the formation of the “train” of ordered fragments.

The digested fragments are released from the parent nucleic acid and continue to move downstream in the sheath fluid in the particular order in which they occurred in the parent nucleic acid from the leading end of the nucleic acid as it moves through a channel, for example, to the trailing end of the nucleic acid (which may be but need not be the order in which they were released from the parent). This will be described in greater detail herein.

Once released from their parent nucleic acid, the digested fragments are allowed to relax their conformations by reducing or eliminating the elongation force(s). Assuming a more relaxed form also causes gaps to form between digested fragments. Such gaps can be increased through further introduction of sheath fluid in the channel. In some instances, intercalator is introduced into the fluid stream at this point and is allowed to bind to the digested, relaxed fragments. (In other instances, the intercalator was combined with the parent nucleic acid prior to cutting, optionally prior to or at the same time as or after binding to RE.) Excess unbound intercalator may or may not be removed. The ordered digested fragments are then passed through an imaging system such as but not limited to a fluorescence microscope in order to measure the intensity such as fluorescence intensity of each relaxed fragment, which is proportional to the amount and thus length of the digested fragment.

The process can be repeated using a different RE, and the results can be used in combination to develop a signature or a more detailed map of the parent nucleic acid.

The ability to maintain the order of the digested fragments, as they exist in the parent nucleic acid from which they came, renders the method superior to prior art methods including PFGE. The fragments are maintained in this order by performing the digestion while the parent nucleic acid (and correspondingly any released fragments) are completely stretched or nearly completely stretched. The ability to manipulate the parent and digested nucleic acids in flow allows for a wider range of manipulations to be performed and more finely controlled. The data so obtained can be used to replace or supplement sequence data obtained using for example PFGE. The approach can be used to analyze simple as well as complex sample and nucleic acid mixtures. It will also yield quantitative information since it analyzes a single nucleic acid at a time as compared to a bulk analysis.

The methods provided herein may be referred to as non-hybridization methods since they do not require hybridization of nucleic acids to each other, such as for example hybridization of a parent nucleic acid to a nucleic acid probe, in order to obtain nucleotide sequence of the parent nucleic acid. Rather the methods provided herein obtain nucleotide sequence of the parent nucleic acid based on the cleavage of the nucleic acid by an RE of known sequence specificity. Additionally, the methods provided herein do not detect RE bound to the parent nucleic acids or their fragments. The RE are therefore not labeled with detectable labels, and nor must they be bound to the nucleic acid fragments while such fragments are being detected. Moreover, the nucleic acid fragments are detected based on signal from bound intercalator. This simplifies the detection system necessary to detect such fragments, since it must only detect signal from the intercalator, rather than signal from intercalator and another agent bound to the fragment, such as for example the RE or a hybridized probe, etc.

The methods provided herein may be performed on single nucleic acids such as single DNA molecules, and are thus referred to as “single molecule analysis” methods. The nucleic acids are typically not fixed or immobile (e.g., conjugated to a support such as a bead or a surface) and rather are in flow in a fluid stream.

Thus, one aspect of this disclosure provides a method for manipulating a nucleic acid in flow comprising (1) digesting an elongated (or stretched) parent nucleic acid, in flow, with a sequence-specific endonuclease, to generate a plurality of digested fragments, (2) maintaining the digested fragments in a linear arrangement in flow that represents the order of the fragments in the parent nucleic acid (from leading end to trailing end), and (3) determining the length of each digested fragment.

In some instances, digestion may be but need not be sequential digestion. As used herein, sequential digestion means that the nucleic acid is digested in an ordered manner from one end (typically the leading end) to the other end (the trailing end), resulting in fragments in a linear order that mirrors the linear arrangement of such sequences in the parent nucleic acid prior to digestion.

In some embodiments, the sequence-specific endonuclease is a restriction enzyme. In some embodiments, the restriction enzyme is a type II restriction enzyme. In some embodiments, the restriction enzyme is a PD . . . D/ExK restriction enzyme.

In some embodiments, the elongated parent nucleic acid is in flow in a microfluidic channel. The microfluidic channel may have a diameter of about 10 microns or greater. In some embodiments, the method is performed in a microfluidic device.

In some embodiments, the length of each digested fragment is determined based on its signal intensity, such as fluorescence intensity. The digested fragments may be stained with intercalator prior to determining their length. In some embodiments, the digested fragments are stained with intercalator after digestion. In some embodiments, the digested fragments are stained with intercalator prior to digestion.

In some embodiments, the digested fragments are relaxed following digestion.

In some embodiments, the parent nucleic acid is elongated using hydrodynamic force. In some embodiments, the parent nucleic acid is elongated using electrophoresis such as gel-free electrophoresis. Electrophoresis can also be used to transport the nucleic acid from the binding condition to the cleaving condition. In some embodiments, a combination of hydrodynamic force and electrophoresis is used to transport the nucleic acids along the channel and/or through the device.

Another aspect of this disclosure provides a method for obtaining sequence information from a nucleic acid comprising (1) incubating a parent nucleic acid with a plurality of REs (of identical type, for example, all are BamHI) under conditions that allow the REs to bind to but not cleave the nucleic acid, (2) elongating the parent nucleic acid with bound REs while in flow, (3) altering the conditions sufficiently to cause the bound REs to cleave the parent nucleic acid, thereby creating a plurality of digested fragments linearly arranged in flow, (4) optionally staining the digested fragments with an intercalator while maintaining the position of each relative to the other digested fragments, and (5) measuring signal intensity, such as fluorescence intensity, of each digested fragment individually in a sequential manner, wherein the fluorescence intensity and detection order of the digested fragments, together with the sequence specificity of the RE yield a map of the parent nucleic acid. In some embodiments, the parent nucleic acid is stained with intercalator prior to, at the same time as, and/or after incubation with the plurality of REs but before digestion.

In some embodiments, the parent nucleic acid is a DNA. The parent nucleic acid may be on the order of 20-1000 kbp in length, or 20-750 kbp in length, or 20-500 kbp in length, or 50-500 kbp in length.

The digested fragments may be on the order of 500 bp to 100 kbp in length, or 1 to 50 kbp in length, or 1 to 20 kbp in length, or 1-10 kbp in length, or 1-7.5 kbp in length, or 1-5 kbp in length.

These and other aspects and embodiments will be described in greater detail herein.

### BRIEF DESCRIPTION OF DRAWINGS

FIG. 1 is a schematic of a plurality of individual parent nucleic acids bound to a plurality (more than one) RE of the same type. The Figure illustrates that the parent nucleic acids in the sample may not be identical, and may not be bound to the same number of REs. The totality of the parent nucleic acids may span the entire genomic DNA of a pathogen, or the entire genomic DNA of a plurality of pathogens. The method identifies the number and location of RE binding/cutting sites on a parent nucleic acid, thereby generating a “map” of those sites per parent nucleic acid. Those maps can then be overlayed to obtain a larger map for a given sample. In addition, the analysis can be performed with different REs (i.e., REs that bind/cut at different locations) sequentially, and the maps generated from each analysis can be overlayed as well for a more detailed genetic map.

FIG. 2 is a schematic of the plurality of parent nucleic acids each bound to REs flowing through a channel of narrowing cross-sectional area. The Figure illustrates that converging channel walls (which lead to cross-sectional area) can be used to apply an extensional strain on the nucleic acids, and this strain elongates the nucleic acids. The Figure also illustrates an embodiment in which the nucleic acids enter the narrowing channel individually. This can be achieved by reducing the concentration of the nucleic acids either prior to loading into the channel or during the movement of the nucleic acids through the channel. Importantly, the fragments are linearly organized and remain in the order in which they were present in the parent nucleic acid. This is achieved by maintaining the parent nucleic acid and the fragments in a completely stretched or nearly completely stretched state during the digestion step.

FIG. 3 is a schematic of a parent nucleic acid moving through a channel having side channels through which is flowed magnesium ions (Mg²⁺) or manganese ions (Mn²⁺), or a combination thereof. Following contact with these ions, the previously intact parent nucleic acid is cleaved into fragments (referred to herein as digested fragments or digested nucleic acids). The RE dissociates from the parent nucleic acid following cleavage. The digested fragments continue to move through the channel in an elongated state. Importantly, the fragments are linearly organized and remain in the order in which they were present in the parent nucleic acid.

FIG. 4 is a schematic of further passage of the digested fragments through the channel and the various modifications the nucleic acids undergo. First, the extensional strain is reduced with the result that the elongated fragments relax and adopt a more condensed or coiled state (illustrated as circles in the Figure). In some instances, the rate of relaxation can be further altered by including viscosity agents in the sheath fluid. The distance between the fragments can also be increased by introducing more sheath fluid into the channel via for example side channels, as illustrated. The distance between the fragments may be increased to ensure that the fragments are recognized and analyzed downstream as single events. Importantly, the fragments are linearly organized and remain in the order in which they were present in the parent nucleic acid.

FIG. 5 is a schematic of intercalation of the relaxed fragments as they flow through the channel. As discussed herein, staining with intercalator may occur prior to binding of RE, at the same time as binding of RE, and/or after binding of RE, as well as after cutting by RE. The Figure provides one embodiment in which intercalator is introduced into the channel through a side channel. The Figure also illustrates a second set of side channels through which sheath fluid, including intercalator, is removed from the channel once intercalation occurs. The length of the channel between these two sets of side channels may vary depending on the time it takes to intercalate the nucleic acid sufficiently. Each nucleic acid will be intercalated to the same extent on average. Importantly, the fragments are linearly organized and remain in the order in which they were cleaved from their parent nucleic acid. Once intercalated and sufficiently separated from each other, each fragment is flowed through an excitation zone which may comprise a laser (to excite the intercalator) and a detection zone (to detect fluorescence emission from the intercalator). The excitation zone and the detection zone may be in the same region.

FIG. 6 is a schematic illustrating a particular embodiment of the invention. Cutting order refers to the order of the fragments as they existed in the parent nucleic acid.

FIG. 7 is a schematic illustrating a particular embodiment of the invention.

FIG. 8 is another schematic illustrating a particular embodiment of the invention. While the Figure refers to staining of DNA with PicoGreen, it is to be understood that other intercalators can be used, including for example SYBERGreen. Intercalators that do not significantly impact DNA structure upon binding to DNA may be used in such embodiments. Such intercalators are known in the art.

FIG. 9 is a photograph of a gel electrophoresis run of lambda DNA mixed with REs ApaI, SmaI and BamHI in binding only buffer. No cutting is observed.

FIG. 10 is a photograph of a gel electrophoresis run of lambda DNA mixed with REs ApaI, SmaI and BamHI in binding buffer (may be referred to herein as “binding only” buffer) spiked with cutting buffer as described in the Examples. The introduction of magnesium via the spiking process results in cutting of the DNA and produces expected digestion maps as shown.

FIG. 11 is a photograph of two tubes, the left tube comprising PicoGreen in binding buffer and the right tube comprising DNA with PicoGreen in the same binding buffer. Enhanced signal from the right tube is apparent.

### DETAILED DESCRIPTION OF INVENTION

Provided herein is a process for high-throughput analysis of nucleic acid samples that involves mapping nucleic acids based on number and relative location of RE binding/cleavage sites. Significantly, the methods provided herein maintain digested fragments in flow in the order in which they are present in a parent nucleic acid. These fragments are maintained in this order until they are analyzed by performing the digestion under conditions that maintain the nucleic acids, whether parent nucleic acids or fragments thereof, in a sufficiently elongated (or stretched) state. The ability to maintain this order provides further information about the relationship of the fragments relative to each other, and allows the parent nucleic acid to be reconstructed more efficiently and accurately. The ability to perform this method on a nucleic acid in flow allows for high-throughput capacity, among other things.

The method derives the following information about a parent nucleic acid: (1) the number of binding/cutting sites for the particular RE used (based on the number of fragments that are detected in a short window of time), (2) the distance between those binding/cutting sites (based on the fluorescence intensity of each detected fragment), and (3) the order of those binding/cutting sites (based on the order in which the fragments are detected).

The method is a significant improvement over prior art methods used to generate maps of genomic DNA. The method does not require fixation of the parent nucleic acid or of its digested fragments. The method analyzes single nucleic acids on an individual basis and therefore it does not require amplification of the original nucleic acid sample. It also does not rely on hybridization of probes as the indicators of sequence. Rather the sequence readouts are derived from the binding/cleavage sites of the REs used.

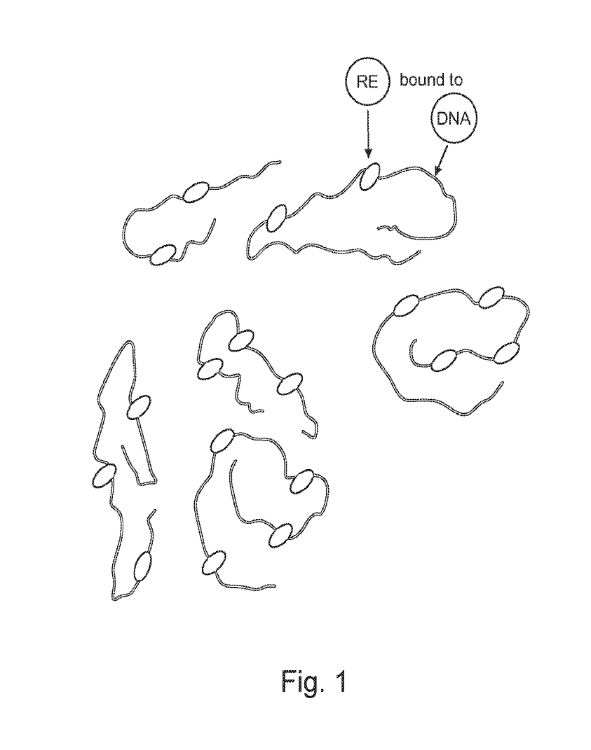

The steps of an embodiment of the method are described in greater detail now. Reference can be made to FIGS. 1-5.

As a first step, a single or a plurality of parent nucleic acids such as parent DNA are mixed with a plurality of REs. The REs may be virtually any RE including those classified as PD . . . D/ExK RE. Significantly, the mixing of the RE and the parent nucleic acid occurs under conditions that promote binding of the RE to the parent nucleic acid but are not conducive to cleavage of the nucleic acid by the RE. This can be accomplished by using calcium ions in the solution instead of magnesium or manganese ions. The mixture is incubated until RE binding to all available specific sites is accomplished. A schematic of parent nucleic acids bound to the REs is provided in FIG. 1. This step may also involve mixing the parent nucleic acids with intercalator, before, during and/or after mixing with the RE.

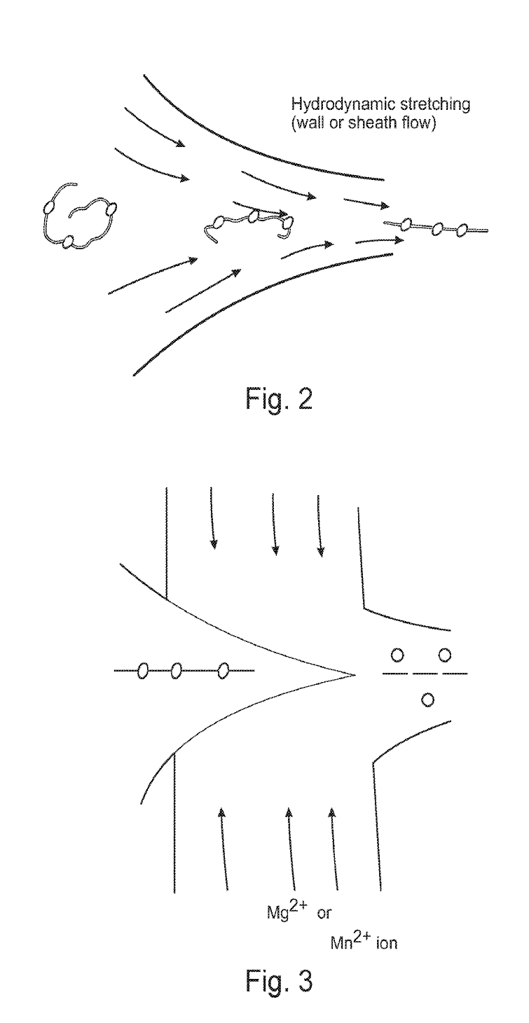

As a second step, the parent nucleic acid is hydro-dynamically stretched. This can be accomplished by modulating the fluid strain rate also known as extensional strain rate. In one embodiment, the fluid strain rate, epsilon, is modulated such that it is nearly equal to or greater than ‘1/tau’ where tau is the longest relaxation time of the parent fragment. The extensional strain rate can be modulated using channel wall geometry or sheath flow velocity (or flow rate) or both. The disclosure contemplates that this and subsequent steps are performed in a micro-fluidic device. FIG. 2 illustrates an embodiment in which the channel wall geometry creates a constriction for flow which in turn increases the fluid velocity and creates an extensional strain rate in flow field. Hydrodynamic focusing and extension of nucleic acids has been described by Wong et al. J. Fluid Mech., 497:55-65, 2003. A completely stretched nucleic acid has an end to end distance that is greater than 0.8 times its contour length. In some instances, the end to end distance of the parent nucleic acid may be less than 0.8 times its contour length, provided such nucleic acid is still sufficiently stretched to render the RE binding and cleavage sites linearized in space.

As a third step, when the parent nucleic acid is at or near a fully stretched state, Mg²⁺ or Mn²⁺ ions are introduced. The introduction of these ions will initiate digestion of the parent nucleic acid. These ions can be introduced into the flow stream for example through a porous wall in the channel and/or by using sheath fluid comprising one or both ions. FIG. 3 illustrates sheath fluid with either Mg²⁺ or Mn²⁺ ions introduced in a manner that causes digestion of the stretched parent nucleic acid to occur in an ordered manner. It is to be understood that digestion may occur in an ordered manner or it may occur in a simultaneous manner or it may occur in a random manner. Digestion is likely to occur so quickly and may therefore appear to be simultaneous or nearly simultaneous. Regardless, the order in which the bound RE cut their respective sites is less important than maintaining the nucleic acids at or near fully stretched states. The fragments will be on the order of 1 kbp (kilo base pairs) or longer. It will be understood that the fragments will be of different lengths, since their lengths will depend on the location of the RE binding/cleavage sites.

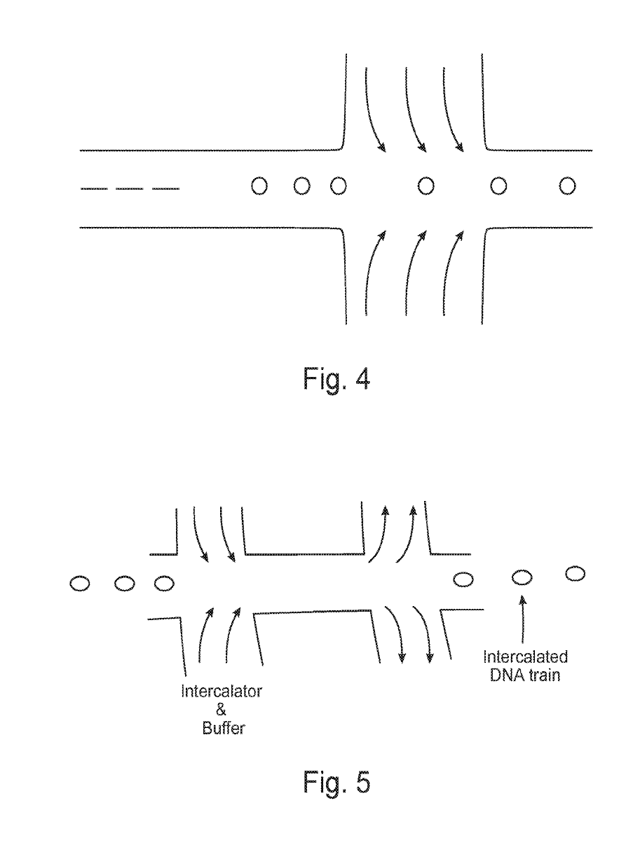

As a fourth step, once digestion has occurred, the digested fragments are now allowed to relax from their stretched state to a more relaxed state by either eliminating the extensional strain rate, epsilon, or by maintaining epsilon at a value that is less than that required to stretch DNA fragments. This process will increase the distance between the digested DNA fragments, thereby creating gaps. These gaps can be increased by introducing sheath fluid as shown in FIG. 4. One or more viscosity agents may be introduced here as well in order to reduce the relaxation rate.

Optionally, as a fifth step, the ordered series (or train) of digested fragments can be intercalated. A pure diffusion process can be used to exchange sample fluid (fluid carrying the nucleic acid fragments) in the channel with a sheath fluid containing an intercalator. This too can be accomplished using channels with a porous wall or by introducing intercalator as shown in FIG. 5.

As a sixth step, the train of intercalated digested and relaxed fragments is passed through an imaging system such as a fluorescence microscope which yields a fluorescence intensity measurement for each fragment that is proportional to length of the digested fragment [ref. 1]. The calculated length of the digested fragment can then be used to estimate the cleavage site of the RE on the parent nucleic acid.

The process can be repeated one or more times to find cleavage sites on another parent nucleic acid, which may be identical to or different from the first analyzed parent nucleic acid. Spacing between parent nucleic acids can be controlled by adjusting the concentration of such fragments when mixed with RE in step 1. The spacing between parent nucleic acids is kept larger than spacing between digested fragments.

The channels and detection regions can be arranged adjacent to each other and processing and detection through each of the channels can occur simultaneously. In this way, the same sample or multiple samples may be processed concurrently. If the same sample is present in a number of channels, then each channel may comprise nucleic acids bound to a different RE.

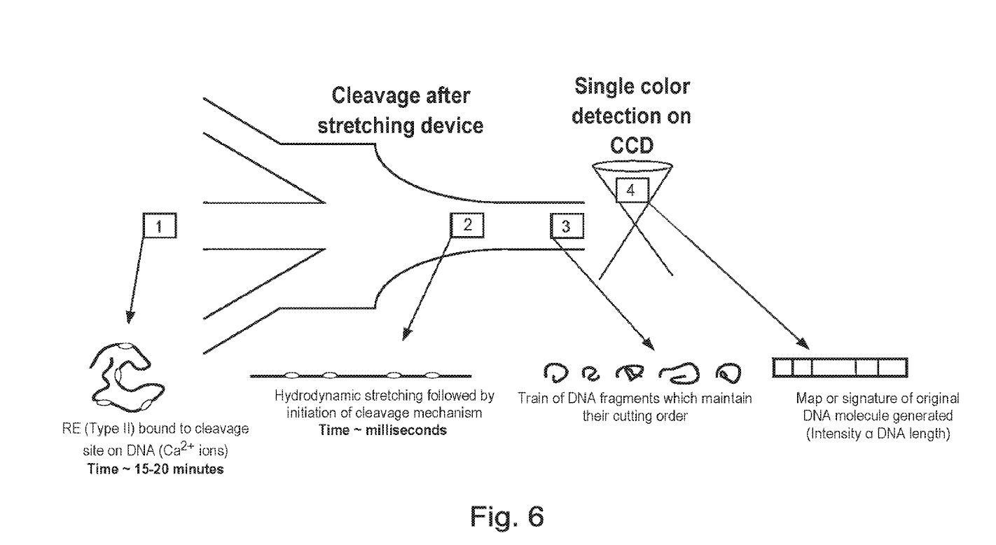

A combination of the foregoing steps is illustrated in FIG. 6. The Figure illustrates one embodiment of the methods provided herein. A type II RE is bound to its cleavage site on the parent nucleic acid in the presence of Ca²⁺ cations (denoted as 1). This binding step may be performed in a microfluidic device (e.g., in a reservoir of such device) or it may be performed separately from the device and the RE-bound nucleic acids can then be loaded into the device. The binding of RE to the nucleic acids may take on the order of about 15-20 minutes, although it is possible that such incubation may be shorter or longer depending on the temperature of the solution, the particular RE used, etc. Such parameters are known to those of ordinary skill in the art. The solution may be referred to as a binding buffer, and it may contain Ca²⁺ cations as discussed herein, as well as buffering agents such as but not limited to Tris, EDTA, and NaCl. It will not contain Mg²⁺ and Mn²⁺ at all or it will not contain these ions at single or combined concentrations that facilitate cleavage of the nucleic acids by the bound RE.

Once the RE-bound nucleic acids are loaded into the microfluidic device, they are hydrodynamically stretched (denoted as 2). The stretching may be accomplished in a number of ways. In the Figure, stretching is accomplished using a two side streams that merge with a middle stream in a microchannel. In this configuration, the flow accelerates in the direction of movement (left to right). The stretching step can take on the order of milliseconds, and may be performed through a change in geometry of the microfluidic channel through which the nucleic acids are travelling. Movement of the nucleic acid as well as stretching of the nucleic acid may be performed using electrophoresis, instead of or in addition to hydrodynamic force. Microfluidic channel diameters may be, on average, greater than 10 microns. The microfluidic device is relatively simple in that it does not contain a post array in order to separate or elongate the nucleic acids. It also does not contain a semi-solid or solid matrix such as a gel. And it does not require any particular surface chemistry. For example, the nucleic acids simply move through the channels and are not attached to the channel walls and accordingly the interior surface of those walls do not comprise any chemical substituents required for binding of nucleic acids.

Once the nucleic acids are stretched, each is individually cut a plurality of times (the plurality dependent on the number of bound RE). Such cutting may start from the leading end of the nucleic acid and finish at the trailing end of the nucleic acid, in some non-limiting embodiments. Alternatively, cutting may occur simultaneously or randomly. The disclosure contemplates all of these possibilities. This can be accomplished by exposing the nucleic acid to a cleaving condition, such as for example Mg²⁺ and/or Mn²⁺ at a single or combined concentration that facilitates cleavage by the bound RE. As the nucleic acid moves through the region of the channel comprising the cleaving condition, it gets cleaved, and its released fragments are maintained in an ordered linear arrangement, with each fragment cleaved from the parent nucleic acid continuing to move downstream in the channel.

Following cleavage, the RE dissociates from the nucleic acid. In addition, the released nucleic acid fragments are no longer elongated (due to a change in channel geometry or a change in sheath fluid dynamics) and are able to assume their native coiled state (denoted as 3). In some instances, a viscosity agent may be used (or may be introduced) to slow the relaxation rate of the nucleic acid fragments. Thus, it is contemplated that the sheath fluid comprises a viscosity agent throughout the process or that a viscosity agent is introduced into the sheath fluid after cutting of the nucleic acid. In the presence of the viscosity agent, the nucleic acid fragments adopt their native coiled state at a slower rate (as compared to in the absence of the viscosity agent), which in some instances is desirable. An example of such a viscosity agent is glycerol. Other viscosity agents are known in the art, including without limitation sucrose, and polymers such as polyethylene glycol (PEG), polyacrylamide or polyvinyl alcohol (PVA) all of which may be provided in aqueous solution forms.

In some instances, the released nucleic acid fragments may be exposed to intercalator following cleavage as they travel downstream through the channel. Once sufficiently intercalated, the nucleic acids are detected in the order in which they are travelling. In other embodiments, the parent nucleic acid may be exposed to intercalator before binding to RE, while binding to RE, and/or after binding to RE, including before cutting with RE.

Detection intends that the signal such as fluorescent signal from each fragment is detected and measured. The amount of fluorescence emitted from each fragment is proportional to the amount of intercalator bound to the fragment which is itself proportional to the length of the fragment. Thus, by measuring the amount of fluorescent signal from each fragment, it is possible to determine the length of such fragment, and thus the distance between two consecutive binding/cleavage sites for the RE that was bound to the nucleic acid. Only a single color (or wavelength) detection system is required because only fluorescence from the intercalator is being detected. Moreover, fragment detection is based on detection of the totality of intercalators bound to each fragment. Since each fragment will be bound to hundreds or thousands of intercalators, the signal from each fragment will be sufficiently strong to be detected. The signal to noise ratio will be much higher than methods that rely on hybridization and detection of signals from single probes. The analysis of a plurality of nucleic acids simultaneously can be achieved using parallel channels and detection zones, as may be accomplished with for example linear detector arrays such as CCD arrays. The process is able to process nucleic acids from greater than 10⁷ cells in about 30 minutes.

It will be apparent that no hybridization occurs and that the method does not ultimately detect the bound RE. It will also be apparent that the method does not involve fixation of nucleic acids, and instead relies on the parent nucleic acids and their digested fragments to move in flow or in solution along a channel such as a microfluidic channel. The nucleic acids are in flow (in movement) throughout the analysis, and thus the methods provided herein are distinguished from “static” methods that fix parent nucleic acids and/or their digested fragments. The channels or other suitable devices are filled with liquids and not gels (i.e., it is a gel-free or sieve-free method). The methods do not require any form of surface derivatization of the channel walls. The methods provided herein also detect nucleic acids in a coiled state. That is, for the methods provided herein, it is important for the nucleic acids to be stretched early on in the process and while they are cleaved by RE. Beyond such cleavage, including while they are being detected and their fluorescence is being measured, the nucleic acids may be and typically are in a relaxed or coiled state.

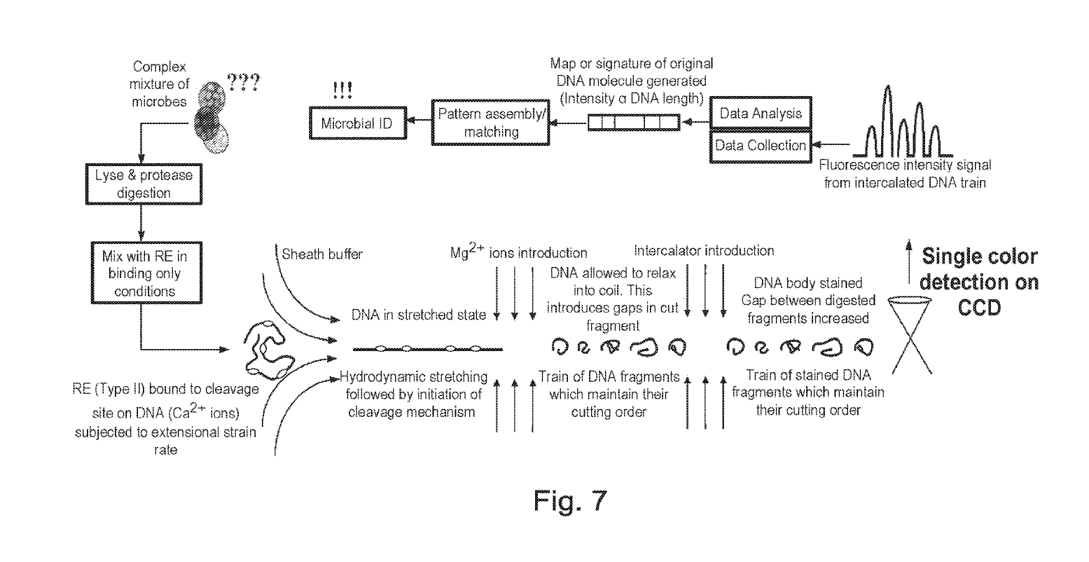

Another embodiment of the disclosure is provided in FIG. 7. This Figure illustrates the various steps starting from a sample having a complex mixture of microbes (or pathogens), lysis and DNA harvest from such sample, binding of the harvested DNA with RE in a binding condition (Ca²⁺ and no Mg²⁺ or Mn²⁺), hydrodynamic stretching of the DNA and exposure of the DNA to the cleaving condition (e.g., introduction of Mg²⁺ ions), allowing the DNA to relax into a coiled state, staining the DNA with intercalator, optionally increasing the distance between digested fragments, detection of the fragments using for example a single color detector CCD, data collection and analysis, creation of a map or signature of the parent DNA (based on the proportional relationship between fluorescence intensity and DNA length), pattern assembly/matching (e.g., arranging various fragments relating to each other, including with overlap between fragments), and optionally identification of one or more microbes in the original complex sample.

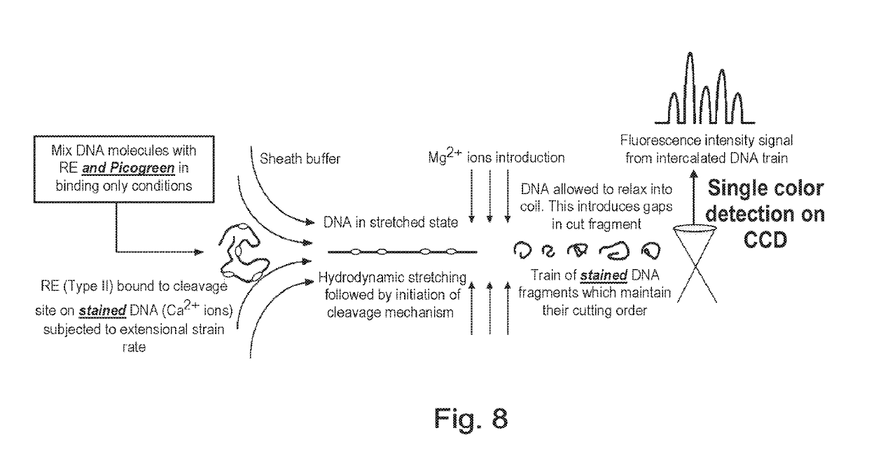

Yet another embodiment of the disclosure is provided in FIG. 8, which is a variation of FIG. 7. This Figure illustrates the various steps starting from mixing the harvested DNA with RE, such as a type II RE, and an intercalator, such as PicoGreen, in a binding condition (e.g., Ca²⁺, no Mg²⁺, no Mn²⁺), hydrodynamic stretching of the DNA and exposure of the DNA to the cleaving condition (e.g., introduction of Mg²⁺ ions), allowing the DNA to relax into a coiled state, optionally increasing the distance between digested fragments, detection of the fragments using for example a single color detector CCD, data collection and analysis. Subsequent steps as shown in FIG. 7 such as creation of a map or signature of the parent DNA (based on the proportional relationship between fluorescence intensity and DNA length), pattern assembly/matching (e.g., arranging various fragments relating to each other, including with overlap between fragments), and optionally identification of one or more microbes in the original complex sample, are also contemplated. FIG. 8 illustrates that the DNA may be labeled with intercalator before and/or during incubation with RE, including optionally under the binding conditions, without significant (if any) impact on RE binding.

As used herein, a nucleic acid in flow means a nucleic acid that is not attached at any point to a solid support. Thus, the nucleic acid moves along in a sheath fluid while the various manipulations and modifications described herein are performed.

### Sample Preparation

The samples being tested may be manipulated in a number of ways. For example, the samples may be washed and spun in order to concentrate cells. In some examples, the samples need not be washed and/or the cells need not be concentrated. Cells within the samples may be cultured prior to nucleic acid harvest or they may be used directly without in vitro culture.

Cells are then lysed using any variety of methods. The resultant cell lysate is protease-treated, and the nucleic acids are released. The nucleic acids may then be sheared, for example by hydrodynamic shearing, for a particular period of time in order to produce parent nucleic acids of about the same size, and optionally in the range of 20-500 kbp (kilo base pairs). Alternatively, the nucleic acids may be cut with a rare cutter RE in order to obtain fragments of a particular length. Examples of such rare cutters include but are not limited to NotI, XbaI and ApaI. The fragments that result after digestion with such rare cutters are mostly in the size range of 20-700 kbp.

### Sequence-Specific Endonucleases Including Restriction Enzymes (REs)

The sequence-specific endonuclease is a nuclease that binds to and cleaves a nucleic acid such as a DNA in a sequence-dependent manner. An example of a sequence-specific endonuclease is a restriction enzyme (RE).

The RE may be a type I RE, a type II RE, a type III RE or a type IV RE. Reference can be made to Pingoud et al. for the details of each RE type. (Pingoud et al. CMLS, Cell. Mol. Life Sci. 62: 685-707, 2005. In some embodiments, the RE is a type II RE. Type II RE tend to cleave within or close to their recognition site and do not require ATP (as do Types I and III) or GTP (as does Type IV). Most type II RE utilize Mg²⁺ for cleavage. Type II REs cleave DNA at defined sites that are 4-8 bp (base pairs) in length. Most type II REs belong to the PD . . . D/ExK family of REs.

Examples of type II RE include but are not limited to ApaI, BamHI, Bgll, Bglll, EcoRI, EcoRV, Muni, PvuII, Haelll, HinPI, Notl, Pmel, SmaI and Eagl. Examples of PD . . . D/ExK REs include BamHI, BglII, BsoBI, Bse634I, Cfr10I, EcoRI, EcoRII, EcoRV, FokI, MunI, and NgoMIV.

In some embodiments, a combination of RE is used. An example of such a combination is ApaI, BamHI and SmaI. The choice of REs may in some instances be governed by the degree of cutting that is desired. In some embodiments, if a combination of REs is used, the combination may include a low frequency (or rare) cutter, a medium frequency cutter, and a high frequency cutter.

It is to be understood that if more than one RE is used to analyze the DNA, the REs may be used simultaneously (i.e., a mixture of such RE is incubated with the DNA) or they may be used individually but in parallel.

### Conditions

The sample fluid may be virtually any fluid through which nucleic acids can travel. At a minimum, typically, it may be an aqueous solution. It may optionally comprise buffering agents, salts with the provisos provided herein, preservatives, and the like.

RE binding relies on diffusion of the nucleic acids and RE in order for RE to find and bind to their binding/cleavage site. This process usually takes about 15-60 minutes to complete. The methods described herein take advantage of the fact that the sequence-specific binding and cleavage activities of REs can be separated in time. That is, under certain conditions, it is possible for a RE to bind to a parent nucleic acid in a sequence-specific manner without cleaving the nucleic acid. The conditions can then be changed in order to cause cleavage by the RE.

REs require a sufficient concentration of magnesium ions (Mg²⁺) or manganese ions (Mn²⁺) to cleave a nucleic acid. In the methods provided herein, the parent nucleic acids are contacted with REs in the absence of Mg²⁺ and Mn²⁺ (or in insufficient amounts or concentrations of one or both of these ions). Under such conditions, the REs are able to bind to the nucleic acids in a sequence-specific manner but they are not able to cleave the nucleic acids. The binding sheath fluid may contain calcium ions (Ca²⁺), although this is not an absolute requirement. The RE may be modified versions of naturally occurring RE, that are able to bind and cut under different conditions. Thus, the nature of the binding and cleaving conditions may vary depending on the nature of the RE used. Conditions that allow REs to bind but not cleave nucleic acids are referred to herein as binding conditions. Conditions that allow nucleic acid-bound REs to cleave nucleic acids are referred to herein as cleaving conditions. The mechanisms by which REs bind in the absence of Mg²⁺ and Mn²⁺ and in the presence of Ca²⁺ and cleave in the presence of Mg²⁺ and/or Mn²⁺ are described in detail in Pingoud et al. CMLS, Cell. Mol. Life Sci., 62:685-707, 2005. The distinction between binding and cleaving conditions and RE activities have also been described by Belkebir and Azeddoug, Microbiol. Res. 168:99-105, 2013 for SepMI and EhoI REs.

Thus it will be understood that in various aspects of this disclosure the nucleic acids are contacted with a plurality of REs (of identical type) in a binding condition thereby allowing the REs to bind to the parent nucleic acids, and then the nucleic acids with REs bound thereto are placed in a cleaving condition thereby allowing the REs to cleave the parent nucleic acids. Once the REs cleave the parent nucleic acids, the REs will dissociate from the nucleic acid.

The binding condition may be a condition that comprises a Mg²⁺ concentration or a Mn²⁺ concentration or a Mg²⁺/Mn²⁺ combined concentration that does not allow cleavage of a nucleic acid. The binding condition may be a condition that lacks Mg²⁺, that lacks Mn²⁺, or that lacks Mg²⁺ and Mn²⁺. The binding condition may be a condition that comprises Ca²⁺ only and no Mg²⁺ or Mn²⁺ ions. Most Type 2 REs inherently show binding only activity in conditions where Ca²⁺ only is present without any Mg²⁺/Mn²⁺ ions.

Some embodiments of this disclosure provide for the use of a combination of RE simultaneously. In these embodiments, the binding and cutting conditions are chosen such that each of the two or more RE bind and cut the DNA with about equal efficiency. Cutting buffers to be used for two or more RE are known in the art and some are available commercially. For example, the Cutsmart® Buffer, available from New England BioLabs, comprises 50 mM Potassium Acetate, 20 mM Tris-acetate, 10 mM Magnesium Acetate, 100 μg/ml BSA, and has a pH 7.9 at 25° C. This buffer provides a suitable cutting condition for most RE, and thus may be regarded as a universal cutting buffer. Cutting conditions of the methods provided herein may similarly comprise about 50 mM Potassium Acetate, 20 mM Tris-acetate, about 10 mM Magnesium Acetate, about 100 μg/ml BSA, and have about a pH 7.9 at about 25° C., as an example. Binding conditions, on the other hand, may be similar to these cutting conditions except that the Magnesium Acetate would be substituted with a calcium salt such as but not limited to Calcium Acetate. Thus, in some embodiments, the binding conditions comprise 50 mM Potassium Acetate, 20 mM Tris-acetate, 10 mM Calcium Acetate, 100 μg/ml BSA, and has a pH 7.9 at 25° C. This buffer provides a suitable binding condition (no cutting) for most RE, and thus may be regarded as a universal binding buffer.

The cleaving condition may be a condition that comprises a Mg²⁺ concentration that allows cleavage of a nucleic acid. The cleaving condition may be a condition that comprises a Mg²⁺ concentration in the range of 5-100 mM. A concentration of about 10 mM Mg²⁺ may be used to activate the cleavage activity of most REs. The cleaving condition may be a condition that comprises a Mn²⁺ concentration that allows cleavage of a nucleic acid. The cleaving condition may be a condition that comprises a Mn²⁺ concentration in the range of 5-100 mM. A concentration of about 10 mM Mn²⁺ may be used to activate the cleavage activity of most REs.

### Intercalators

The nucleic acid fragments are labeled with intercalators. As used herein, intercalators are compounds that bind nucleic acids in a substantially sequence-independent manner. Examples include phenanthridines and acridines (e.g., ethidium bromide, propidium iodide, hexidium iodide, dihydroethidium, ethidium homodimer-1 and -2, ethidium monoazide, and ACMA); indoles and imidazoles (e.g., Hoechst 33258, Hoechst 33342, Hoechst 34580 and DAPI); cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red); and miscellaneous stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

In some embodiments, the intercalator is PicoGreen or SYBRGreen, both of which are commercially available. These and other intercalators may be used to label samples having a wide range of DNA concentrations, and thus such intercalators are characterized as being less sensitive to DNA concentration, while still being sufficiently fluorescent. When such intercalators are used, it is not absolutely required that the DNA concentration of each sample be known and/or standardized. In some embodiments, such intercalators may be used in a micromolar range, including from example from about 0.5 to 1.5 micromolar, or about 0.7 to 1.3 micromolar, or about 0.7 to 1 micromolar. In some embodiments, the intercalator is PicoGreen and it is used at a concentration of about 0.8 micromolar. PicoGreen reportedly can be used to stain DNA concentrations in the range of 0.5-10,000 ng/ml stoichiometrically. Larson et al. Cytometry, 2000, 41:203-208.

A further advantage of certain intercalators including PicoGreen and SYBERGreen is their ability to bind to DNA without inducing structural changes in the bound DNA. Schafer et al. Single Mol. 2000, 1:33-40. Since the intercalator does not induce structural changes in the DNA, it also does not impact binding of the RE to the DNA. Accordingly, labeling of a nucleic acid sample with such an intercalator can occur before and/or during binding of the RE.

Still a further advantage of certain intercalators including PicoGreen and SYBERGreen is the ability to function even in relatively high salt conditions such as those used in RE digestion protocols.

The ability to determine size of nucleic acids based on fluorescent signal from bound intercalators is described by Huang et al. Nucleic Acids Research, 24(21):4202-4209, 1996.

### Device

The method may be performed in a microfluidic device. In one embodiment, an exemplary device may comprise: (1) an inlet, optionally with an adjacent reservoir, (2) a first microfluidic channel downstream of the inlet and optional reservoir, the channel having (i) a first region having a geometry that imposes upon polymers moving therethrough an extensional strain rate, epsilon, that is about equal to or greater than 1/tau where tau is the longest relaxation time of the polymers (referred to as an elongation region), and (ii) a second region having parallel walls and that does not impose an extensional strain rate on a polymer moving therethrough (referred to as a relaxation region), (3) a first, second and third set of side channels located along the length of the second region of the microfluidic channel, and (4) a detection zone within the second region. In another embodiment, the device may comprise electrodes at either end in order for electrophoretic movement of negatively changed DNA to occur through the device and channel(s).

### Applications

The methods provided herein can be used to perform detailed genomic DNA mapping of cultured cells. The cells may have been provided as a sample such as a microbiome sample from a normal (healthy) subject or from a subject having or suspected of having an infection. The microbiome sample may be an environmental sample such as a soil sample or a water sample. The sample may a bodily sample such as a urine sample, a blood sample, a sputum sample, or a bowel sample. In the case of a blood sample, the sample may be analyzed for the presence of rare circulating cells such as cancer cells. The sample may be a food sample or a sample derived from a food source. The sample may be a seed sample or a plant that is suspected of being genetically engineered.

The methods may be used to identify one or more or all known pathogens in a sample. In this embodiment, it is contemplated that a genomic map is created and compared to existing genomic maps of known pathogens. The existing genomic maps may be maps generated using other technologies such as PFGE or they may be theoretical maps obtained from knowledge of sequence specificity of an RE and sequence data collected from whole genome sequencing of particular pathogens. Alternatively, the existing genomic maps may be generated using the methodology of this disclosure as applied to pure samples of known pathogens.

The methods may be used to identify variants or mutants of known pathogens. In this latter aspect, it is contemplated that a genomic map is created and compared to existing genomic maps of known pathogens. Newly generated genomic maps that are closely related to existing genomic maps may represent mutants of known pathogens.

The genomic maps obtained using the methods described herein may be used instead of or as a supplement to existing genomic maps generated using other technologies such as but not limited to PFGE.

The genomic maps can then be used to assemble sequence information that is obtained from short read sequences, as is typically obtained from next-generation sequencing technologies such as but not limited to Illumina.

Each patent, patent application and reference cited herein is incorporated by reference in its entirety.

### EXAMPLES

### Example 1. Binding and Cutting Conditions

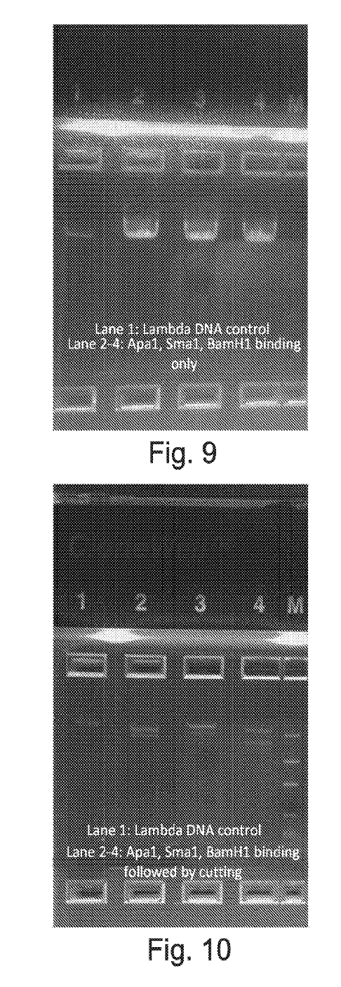

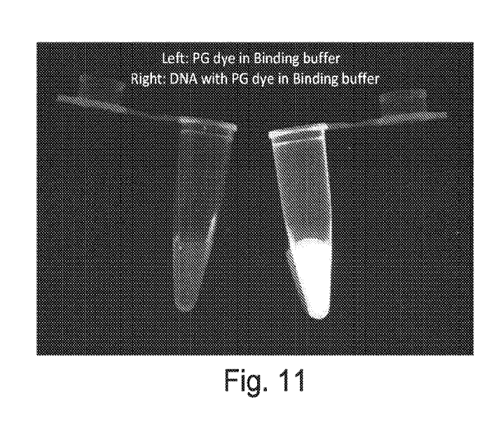

Lambda DNA and RE were mixed in binding (only) buffer conditions (i.e., 50 mM Potassium Acetate, 20 mM Tris-acetate, 10 mM Calcium Acetate, 100 μg/ml BSA, and has a pH 7.9 at 25° C.). FIG. 9 confirms that in these conditions no cutting of DNA molecule occurs as seen from gel electrophoresis. Lane 1 contains lambda DNA control, and lanes 2-4 contain ApaI, SmaI and BamHI respectively. As a control, a portion of the DNA/RE mixture was spiked with 10× Cutsmart® Buffer for a final concentration of 1× Cutsmart® Buffer and 0.1× binding (only) buffer. This produced the expected digestion maps on gel electrophoresis as shown in FIG. 10. Lane 1 contains lambda DNA control, and lanes 2-4 contain ApaI, SmaI and BamHI respectively, wherein the samples in lanes 2-4 were first incubated with binding only buffer and then incubated with cutting buffer (i.e., the spiked buffer described above).

### Example 2. Intercalator Staining Under Binding Conditions

PicoGreen staining of a DNA-RE mixture was performed in binding (only) buffer conditions. FIG. 11 shows PicoGreen stained DNA in the presence of binding only buffer (right tube) and PicoGreen and binding only buffer control (left tube). PicoGreen provides enhanced fluorescence intensity in the presence of DNA in the binding only buffer condition. The tubes are illuminated by blue light and an orange filter is used to filter fluorescence signal onto an iPhone camera.

1. A method for manipulating a nucleic acid in flow comprising
digesting an elongated parent nucleic acid, in flow, with a sequence-specific endonuclease to generate a plurality of digested fragments,
maintaining the digested fragments in a linear arrangement in flow that represents the order the fragments in the parent nucleic acid, and
determining the length of each digested fragment.

2. The method of claim 1, wherein the sequence-specific endonuclease is a restriction enzyme.

3. The method of claim 2, wherein the restriction enzyme is a type II restriction enzyme.

4. The method of claim 2, wherein the restriction enzyme is a PD . . . D/ExK restriction enzyme.

5. The method of claim 1, wherein the elongated parent nucleic acid is in flow in a microfluidic channel.

6. The method of claim 1, wherein the length of each digested fragment is determined based on its fluorescence intensity.

7. The method of claim 1, wherein the digested fragments are stained with intercalator prior to determining their length.

8. The method of claim 1, wherein the digested fragments are stained with intercalator after digestion.

9. The method of claim 1, wherein the digested fragments are relaxed following digestion.

10. The method of claim 1, wherein the parent nucleic acid is elongated using hydrodynamic force.

11. The method of claim 1, wherein the parent nucleic acid is elongated using electrophoresis.

12. A method for obtaining sequence information from a nucleic acid comprising
incubating a parent nucleic acid with a plurality of restriction enzymes under conditions that allow the restriction enzymes to bind to but not cleave the nucleic acid,
elongating the parent nucleic acid with bound restriction enzymes while in flow,
altering the conditions sufficiently to cause the bound restriction enzymes to cleave the parent nucleic acid, thereby creating a plurality of digested fragments linearly arranged in flow,
staining the digested fragments with an intercalator while maintaining the position of each relative to the other digested fragments, and
measuring fluorescence intensity of each digested fragment individually in a sequential manner,
wherein the fluorescence intensity and detection order of the digested fragments, together with the sequence specificity of the restriction enzyme yield a map of the parent nucleic acid.
